(19) United States
(12) Patent Application Publication
(10) Pub. No.: US 2021/0189330 A1
(43) Pub. Date: Jun. 24, 2021

(54) INDUCED TOTIPOTENT STEM CELLS AND METHODS FOR MAKING AND USING THE SAME

(71) Applicant: THE J. DAVID GLADSTONE INSTITUTES, A TESTAMENTARY TRUST ESTABLISHED UNDER THE WILL OF J. DAVID GLADS, San Francisco, CA (US)

(72) Inventors: Cody W. Kime, San Francisco, CA (US); Kiichiro Tomoda, San Francisco, CA (US)

(21) Appl. No.: 16/493,877

(22) PCT Filed: Mar. 22, 2018
(86) PCT No.: PCT/US2018/023712

§ 371 Date: Sep. 13, 2019

Related U.S. Application Data
(60) Provisional application No. 62/475,606, filed on Mar. 23, 2017.

(51) Int. Cl. C12N 5/0735

(52) U.S. Cl. CPC C12N 5/0606; C12N 2501/155; C12N 2501/235; C12N 2513/00; C12N 2501/999; C12N 2506/02; C12N 2500/38

(57) ABSTRACT

The present disclosure provides induced totipotent stem cells as well as methods of making and using the same.

### PRIORITY CLAIM

This application claims priority to U.S. Provisional Application Ser. No. 62/475,606 which was filed on Mar. 23, 2017, the entire contents is incorporated herein by reference and relied upon.

### BACKGROUND

A few days after fertilization, mammalian embryos form a structure called the blastocyst. The blastocyst consists of three distinct parts, an outer layer of trophoblast cells, the pluripotent stem cells (PSCs) of the inner cell mass, and a blastocyst cavity (or blastocoel) inside the outer layer. Once the blastocyst has implanted in the uterus, cells of the inner cell mass develop to the embryo proper with the support of decidua induction by trophoblast cells.

PSCs are characterized as embryonic cells that can give rise to the animal proper. While numerous types of cells, including oocytes, can be differentiated in vitro from PSCs, PSCs do not contribute to the extraembryonic structures from the blastocysts trophectoderm lineage, which induce mesometrium implantation, decidua formation, and yield the placenta. On the other hand, totipotent cells have the ability to form all of the early embryonic lineages, both the cells of the embryo proper as well as trophectoderm lineage. To date, regenerative medicine has hinged upon applications using in vitro PSCs that give rise to the animal proper, but require donor blastocysts or chimerism for extraembryonic support in utero.

Despite the immense clinical and research potential of generating totipotent cells in vitro, no reliable methods have been reported to date for making such cells or for generating function blastocyst structures from ex vivo cells.

### SUMMARY

This disclosure is predicated on the discovery of novel methods for producing totipotent and/or totipotent-like stem cells from non-totipotent cells.

One aspect of the disclosure provides in vitro methods of producing mammalian totipotent and/or totipotent-like stem cells comprising: obtaining a cell population of non-totipotent cells and providing the cell population with an amount of a first conversion media, thereby producing mammalian totipotent and/or totipotent-like stem cells from the non-totipotent cells.

In some embodiments, the non-totipotent cells are selected from the group consisting of a pluripotent stem cell, an epiblast stem cell, an adult stem cell, and a fibroblast, or a combination thereof.

In some embodiments, the pluripotent stem cell is a nave pluripotent stem cell or a primed pluripotent stem cell.

In some embodiments, the totipotent or totipotent-like stem cells can contribute to both extraembryonic and embryonic lineages.

In some embodiments, the totipotent or totipotent-like stem cells express Cdx2, YAP, Hex, Oct4, H3R2me2, Prdm14, H3K4me2, Mouse Retroelement MuERV-L/MERVL, XaXa-GFP, or any combination thereof.

In some embodiments, the mammalian totipotent or totipotent-like stem cells are human totipotent or totipotent-like stem cells.

In some embodiments, at least 0.005%, at least 0.05%, at least 0.5%, at least 1%, at least 5%, or more of the non-totipotent cells are converted to totipotent or totipotent-like stem cells.

In some embodiments, prior to providing the cell population with an amount of a first conversion media the cell population is provided an amount of reversion media.

In some embodiments, the reversion media comprises BMP4, LIF, a LPAR agonist, ascorbic acid, or any combination thereof.

In some embodiments, the first conversion media comprises BMP4, ascorbic acid, a Smad inhibitor, or any combination thereof. In some embodiments, the Smad inhibitor is SB431542. In some embodiments, the first conversion media comprises no or substantially no LIF and/or LPAR agonist.

In some embodiments, the cell population is cultured in the first conversion media for about 1 to about 10 days. In some embodiments, the cell population is cultured in the first conversion media for about 4 days.

In some embodiments, the methods further comprise culturing the cell population with an amount of a second conversion media. In some embodiments, the second conversion media comprises LIF, an LPAR agonist, ascorbic acid, or any combination thereof. In some embodiments, the second conversion media comprises no or substantially no BMP4 and/or SMAD inhibitor.

In some embodiments, the LPAR agonist is 1-oleoyl-2-methyl-sn-glycero-3-phosphothionate (OMPT), lysophosphatidic acid (LPA), or any combination thereof.

In some embodiments, the second conversion media is provided after the first conversion media. In some embodiments, the second conversion media is provided for about 1 day to about 5 days. In other embodiments, the second conversion media is provided for about 3 days.

In some embodiments, the methods further comprise producing a morula-like hemisphere from the mammalian totipotent and/or totipotent-like stem cells. In some embodiments, the methods further comprise producing a blastocyst-like hemisphere from the mammalian totipotent and/or totipotent-like stem cells. In other embodiments, the methods comprise producing an induced blastocyst-like structure (iBC) from the mammalian totipotent and/or totipotent-like stem cells. In some embodiments, cells of the iBC express Troma-I, Oct4, nuclear Cdx2, YAP, or any combination thereof. In some embodiments, the iBC is an isogenic iBC.

In some embodiments, the producing steps are performed in vitro or in vivo. In some embodiments, in at least a portion of the steps are carried out on a cell attachment substrate and/or in a low attachment plate.

In some embodiments, the methods further comprise transplanting the iBC into a pseudopregnant mouse. In some embodiments, the iBC induces decidualization.

In some aspects, provided are methods of maintaining mammalian totipotent or totipotent like cells in vitro.

In some aspects, provided are isolated totipotent or totipotent-like cell prepared according to any of the embodiments detailed and described herein.

In some aspects, provided are aggregates of the isolated totipotent or totipotent-like cells according to any of the embodiments detailed and described herein. In some embodiments, the aggregate is a 2-cell, 4-cell, 8-cell, 16-cell, 32-cell, 64-cell aggregate of totipotent or totipotent-like cells.

In some aspects, provided herein are methods of producing tissue and/or organs from the in vitro derived totipotent or totipotent-like cells according to any of the embodiments detailed and described herein. In some embodiments, the tissue or organ is a patient-specific tissue or organ. In some embodiments, at least a portion of the steps are performed in vitro. In other embodiments, at least a portion of the steps are performed in vivo.

### DESCRIPTION OF THE DRAWINGS

FIG. 1 depicts a representative low magnification image of blastocyst-like hemispheres on Conversion Day 7 according to an embodiment of the present disclosure. Xa/XaGFP (active) overlay indicates naïve-like stem cells. Open Arrow=Morula-like Hemisphere; Solid Arrow=Blastocyst-like Hemisphere.

FIG. 2A and FIG. 2B depict representative high magnification images of blastocyst-like hemispheres on Conversion Day 7 according to an embodiment of the present disclosure. Xa/XaGFP (active) overlay indicates naïve-like stem cells.

FIG. 3 shows representative images of blastocyst-like hemispheres generated according to an embodiment of the present disclosure. FIG. 3A shows a representative bright field image of an early blastocyst-like hemisphere overlaid with fluorescent images showing Nanog expressed in non-flattened GFP negative cells. FIG. 3B shows a representative image of late blastocyst-like hemisphere with Nanog restricted to the GFP positive cells. FIG. 3C shows a representative fluorescent images showing Troma-I (Krt8)-positive cells, a marker for trophectoderm lineage cells, surrounding the blastocoel-like space and oversized Xa/Xa-GFP positive, Nanog positive, polar mass.

FIG. 4 is a representative image of mouse epiblast stem cells (mEpiSCs) undergoing conversion to generate induced blastocyst-like cells (iBCs) according to an embodiment of the present disclosure on Conversion Day 1, approximately 24 hours after the addition of the first conversion media. mEpiSCs are observed growing with some SMAD inhibitor toxicity killing cells. Some cells remain in colony-like clusters, while others begin to appear flattened at the edges.

FIG. 5A is a representative 4× magnification of cells undergoing conversion according to an embodiment of the present disclosure on Conversion Day 2, approximately 48 hours after the addition of a first conversion media.

FIG. 5B is a representative 32× magnification of cells undergoing conversion according to an embodiment of the present disclosure on Conversion Day 2, approximately 48 hours after the addition of a first conversion media. Arrows identify light refractive clusters that infrequently activate MERVL totipotency reporter and sometimes reactivate Xi to Xa in female mEpiSC at later time points.

FIG. 6 is a representative image of cells undergoing conversion according to an embodiment of the present disclosure on Conversion Day 3, approximately 72 hours after the addition of the first conversion media.

FIG. 7A and FIG. 7B are representative images of cells undergoing conversion according to an embodiment of the present disclosure on Conversion Day 4.

FIG. 8 are representative images showing the co-expression of a totipotency reporter (MERVL) (FIG. 8A and FIG. 8D) and Xa/Xa-GFP expression (FIG. 8B and FIG. 8E), colocalized in the same cells (FIG. 8C and FIG. 8F) on Conversion Day 4 of the experiment. MERVL and Xa/Xa-GFP expression are hallmarks of totipotency.

FIG. 9 is a representative image of cells undergoing conversion according to an embodiment of the present disclosure on Conversion Day 5. Arrows identify possible sources of totipotent-like or iBC forming clusters.

FIG. 10 is a representative image of late-cleavage-stage like cells derived according to an embodiment of the present disclosure expressing the MERVL totipotency reporter FIG. 10A shows a representative image of late-cleavage stage like cells expressing MERVL. FIG. 10B shows a representative bright field image of late-cleavage stage like cells.

FIG. 11 depicts modified conditions for release of early embryo-like structures into suspension. FIG. 11A shows panels representative images of an embryo-like iBC released from the plate: TOP LEFT=late morula like iBC 2 days post-release, TOP RIGHT=late blastocyst like iBC 3 days post-release, BOTTOM LEFT=late blastocyst-like iBC paused growing 4 days post release, and BOTTOM RIGHT=8-cell like aggregate 5 days post release. Scale bars=50 μm. FIG. 11B shows a pool of isolated iBCs for sourcing to transfer to pseudopregnant mice. Scale bar=200 μm. FIG. 11C is a representative image of an iBC in culture expressing EOS::DSRED pluripotency marker in ICM-like region. Scale bar=100 um.

FIG. 12 is a representative immunohistochemical image of an iBC-like structure produced according to an embodiment of the present disclosure and showing expression of the extraembryonic lineage marker (Troma-I) and a pluripotency marker (Oct3/4), and nuclear DNA (Hoechst33342).

FIG. 13 a schematic representation of the implantation experiments performed according to an embodiment of the present disclosure. FIG. 13A shows an iBC and Control embryo co-transfer experiment diagram. Calculated deciduae count frequencies are shown as the number observed in excess of control embryos compared to iBCs transferred and the total number of observed deciduae from all co-transfer experiments compared to the number of control embryos transferred. FIG. 13B is a Single Source transfer experiment diagram showing the frequency of deciduae formation in uterus horns with respect to single sources of embryoid bodies (EB), iBCs, or Control Embryos.

FIG. 14 demonstrates that iBCs generated according to an embodiment of the present disclosure induce decidualization and at least partially develop in utero. FIG. 14A is a representative image of an H&E stained embryo in deciduae for positive control H2B-EGFP E6.5 embryo. FIG. 14C is a representative immunohistochemistry staining for co-transfer deciduae showing positive control H2B-EGFP E6.5 embryo with anti-GFP antibody, Troma-I, and DNA. FIG. 14B is a representative image of an H&E stained excess decidua from co-transfer with apparent non-decidua tissue. FIG. 14D is a representative immunohistochemistry staining of the excess co-transfer decidua that did not stain with anti-GFP antibody, but retained Troma-I positive cells, and DNA. This tissue shows Troma-I positive cells at the center lacking H2B-GFP (signal enhanced to show only background present). Scale bars=100 um. FIG. 14F shows results of iBC-only transfer to uterus observed with several induced deciduae at E7.5, dissected and prepared for cryosection and H&E stained to reveal decidua (FIG. 14E, scale bar=500 um) with evidence of resorption including high presence of granulocytes and reduced embryonic cavity with disfigured trophectoderm and embryonic tissue morphology (FIG. 14G, scale bar=100 um).

FIG. 15 is a schematic representation of a method for generating iBCs according to an embodiment of the present disclosure.

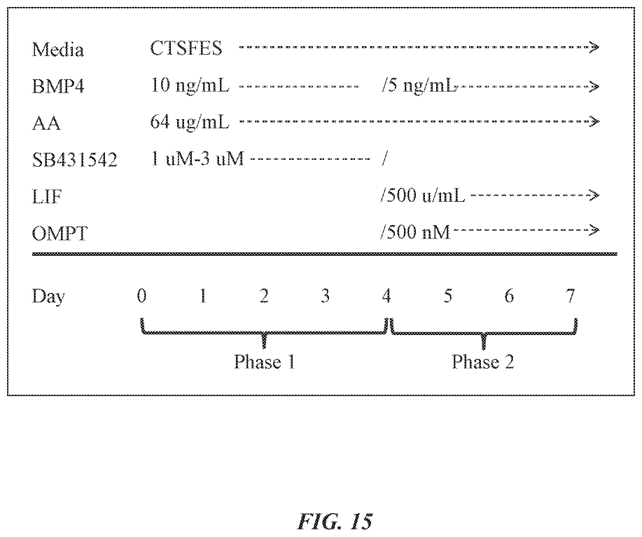

### DETAILED DESCRIPTION

The present disclosure is predicated on the discovery that totipotent and/or totipotent-like stem cells can be induced using a novel in vitro culture technique. Achieving totipotency from non-totipotent cells (e.g., progenitive isogenic stem cells) may circumvent need for artificial placenta development or polygenic cells for use in regenerative medicine. Using these induced totipotent stem cells in conjunction with recent advances genome editing technology may also provide emergent rapid platforms for recombinant mouse production. Advancing this biology among emergent technologies in ectogenesis from genetically-engineered, developmentally incapacitated non-person PSCs should enable a placental 3D organogenic environment that may better produce para-embryonic 3D organs for patient transplant and exclude the need for animal hosts (Unno et al., 1993; Ozone et al., 2016).

It is to be understood that this disclosure is not limited to particular embodiments described, as such may, of course, vary. It is also to be understood that the terminology used herein is for the purpose of describing particular embodiments only, and is not intended to be limiting, since the scope of this disclosure wilt be limited only by the appended claims.

The detailed description of the disclosure is divided into various sections only for the reader's convenience and disclosure found in any section may be combined with that in another section. Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated by reference to disclose and describe the methods and/or materials in connection with which the publications are cited.

All numerical designations, e.g., pH, temperature, time, concentration, and molecular weight, including ranges, are approximations which are varied (+) or (−) by increments of 0.1 or 1.0, where appropriate. It is to be understood, although not always explicitly stated that all numerical designations are preceded by the term “about.” It also is to be understood, although not always explicitly stated, that the reagents described herein are merely exemplary and that equivalents of such are known in the art.

It must be noted that as used herein and in the appended claims, the singular forms “a”, “an”, and “the” include plural referents unless the context clearly dictates otherwise. Thus, for example, reference to “a pluripotent stem cell” includes a plurality of pluripotent stem cells.

### Definitions

As used herein the following terms have the following meanings:

The term “about” when used before a numerical designation, e.g., temperature, time, amount, concentration, and such other, including a range, indicates approximations which may vary by (+) or (−) 10%, 5% or 1%.

Also as used herein, “and/or” refers to and encompasses any and all possible combinations of one or more of the associated listed items, as well as the lack of combinations when interpreted in the alternative (“or”).

“Comprising” or “comprises” is intended to mean that the compositions, for example cell culture media, and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this invention.

The term “stem cell” refers to a cell that is in an undifferentiated or partially differentiated state and has the capacity to self-renew and to generate differentiated progeny. Self-renewal is defined as the capability of a stem cell to proliferate and give rise to more such stem cells, while maintaining its developmental potential (i.e., totipotent, pluripotent, multipotent, etc.). The term “somatic stem cell” is used herein to refer to any stem cell derived from non-embryonic tissue, including fetal, juvenile, and adult tissue. Somatic stem cells have been isolated from a wide variety of adult tissues including blood, bone marrow, brain, olfactory epithelium, skin, pancreas, skeletal muscle, and cardiac muscle. Exemplary naturally occurring somatic stem cells include, but are not limited to, mesenchymal stem cells and hematopoietic stem cells. In some embodiments, the stem or progenitor cells can be embryonic stem cells. As used herein, “embryonic stem cells” refers to stem cells derived from tissue formed after fertilization but before the end of gestation, including pre-embryonic tissue (such as, for example, a blastocyst); embryonic tissue; or fetal tissue taken any time during gestation, typically but not necessarily before approximately 10-12 weeks gestation. Most frequently, embryonic stem cells are pluripotent cells derived from the early embryo or blastocyst. Embryonic stem cells can be obtained directly from suitable tissue, including, but not limited to human tissue, or from established embryonic cell lines.

The term “totipotent” refers to a cell that can give rise to any tissue or cell type in the body as well as extraembryonic tissue, such as the placenta. “Pluripotent” cells can give rise to any type of cell in the body. Cells that can give rise to a smaller or limited number of different cell types are generally termed “multipotent.” Thus, totipotent cells differentiate into pluripotent cells that can give rise to most, but not all, of the tissues necessary for fetal development. Pluripotent cells undergo further differentiation into multipotent cells that are committed to give rise to cells that have a particular function. For example, multipotent hematopoietic stem cells give rise to the red blood cells, white blood cells and platelets in the blood.

The term “pluripotent” as used herein refers to a cell with the capacity, under different conditions, to differentiate to cell types characteristic of all three germ cell layers (i.e., endoderm (e.g., gut tissue), mesoderm (e.g., blood, muscle, and vessels), and ectoderm (e.g., skin and nerve). Pluripotent cells are characterized primarily by their ability to differentiate to all three germ layers, using, for example, a nude mouse teratoma formation assay. Pluripotency is also evidenced by the expression of embryonic stem cell (ESC) markers, although the preferred test for pluripotency is the demonstration of the capacity to differentiate into cells of each of the three germ layers by, for example, an in vitro differentiation assay, and/or chimera formation. Two phases of pluripotency can exist, namely, a naïve state and a primed state.

As used herein the term “isolated” with reference to a cell, refers to a cell that is, at least partially, in an environment different from that in which the cell naturally occurs, e.g., where the cell naturally occurs in a multicellular organism, and the cell is removed from the multicellular organism, the cell is “isolated.” For example, an isolated cell is a cell that is separated from tissue or cells of dissimilar phenotype or genotype.

### Cells

This disclosure is predicated on the discovery of a novel method for producing a totipotent or totipotent-like stem cell from a non-totipotent cell.

As used herein a “non-totipotent cell” refers to a cell that lacks the ability to produce both extraembryonic cell types and embryonic cell types. A non-totipotent cell therefore is of lesser potency to differentiate than a totipotent stem cell. Cells of lesser potency can be, but are not limited to pluripotent stem cells (PSCs), somatic stem cells, tissue specific progenitor cells, primary or secondary cells. Without limitation, a somatic stem cell can be a hematopoietic stem cell, a mesenchymal stem cell, an epithelial stem cell, a skin stem cell or a neural stem cell. A tissue specific progenitor refers to a cell devoid of self-renewal potential that is committed to differentiate into a specific organ or tissue. A primary cell includes any cell of an adult or fetal organism apart from egg cells, sperm cells and stem cells. Examples of useful primary cells include, but are not limited to, skin cells, bone cells, blood cells, fat cells, cells of internal organs and cells of connective tissue. A secondary cell is derived from a primary cell and has been immortalized for long-lived in vitro cell culture. The term “totipotent like” refers to a cell that has most, but not all, of the characteristics of a totipotent cell, for example, production of an isogenic blastocyst that is unable to generate an embryo, fetus, or offspring. The terms “bi-directional” and/or “bi-potential” embryonic stem cells are also sometimes used to describe these cells.

In some embodiments, the non-totipotent cell is selected from the group consisting of a pluripotent stem cell (PSC), an epiblast stem cell, an adult stem cell, and a fibroblast.

The PSCs of the present disclosure include any pluripotent stem cell, for example, an embryonic stem cell (ESC), an epiblast stem cell (EpiSC), an embryonic germ cell (EGC), and an induced pluripotent stem cell (iPSC). In some embodiments, the PSC is a naïve PSC. In other embodiments, the PSC is a primed PSC. Any method known to one of skill in the art for generating naïve PSCs can be used. WO 2016/179243, US Publication No. 20150037883, US Publication No. 20140315301, US Publication No. 20110088107, and Theunissen et al. (2014) Cell Stem Cell, the disclosures of which are hereby incorporated by reference herein in their entirety, describe methods for generating nave stem cells.

Induced PSCs can be generated from numerous types of non-pluripotent stem cells (e.g. fibroblasts, hepatocytes, epithelial cells) by either genetic (e.g., retroviral, adenoviral, episomal), protein, modified RNAs, or chemical manipulation to promote the expression of exogenous factors such as Oct4, Sox2, c-Myc, Klf4 (Yamanaka et al. (2006) Cell 126(4):663-676; Yamanaka et al. (2007) Cell Stem Cell 1(1):39-49; Takahashi et al. (2007) Cell 131:861-872) or endogenous genes using dCas/CRISPR methods (Liu et al. (2018) Cell Stem Cell 22(2):252-261).

The non-totipotent cells of the present disclosure may be derived from a mammal, including humans, non-human primates, murines (i.e., mice and rats), canines, felines, equines, bovines, ovines, porcines, caprines, etc. In some embodiments, the mammalian non-totipotent cells are human cells. In other embodiments, the mammalian non-totipotent cells are non-human mammalian cells.

In some embodiments, a totipotent stem cell can be identified as a cell that can contribute to both extraembryonic and embryonic lineages. Totipotent or totipotent-like stem cells can express Cdx2, YAP, Hex, Oct4, H3R2me2, Prdm14, H3K4me2, Mouse Retroelement MuERV-L/MERVL, Xa/Xa-GFP, or any combination thereof.

In some embodiments, the non-totipotent cell is a modified cell (e.g., genetically modified) to express at least one exogenous factor. The exogenous factor can be any factor known to one skilled in the art. Non-limiting examples of factors include inhibitors of miR34a (Choi et al (2017) Science), inhibitors of mTOR, inhibitors of chromatin assembly (e.g., CAF-1), exogenous gene induction of Dux/Dux4 (Hendrickson et al (2017) Nature Genetics 49(6):925-934. In some embodiments, the non-totipotent cell is not modified to express any exogenous factors.

### Media

The present disclosure provides cell culture media for converting non-totipotent cells into totipotent cells.

In one embodiment, the disclosure provides a first conversion media (“first conversion media” is used interchangeably with “Phase 1”). The first conversion media comprises a basal media and at least one additive (i.e., agent). In some embodiments, the basal media (“CTSFES media”) comprises at least one of DMEM/F12-Glutamax, Neurobasal medium, N2 supplement (Thermo Fisher Scientific, Carlsbad, Calif., USA), B27 supplement (Thermo Fisher Scientific, Carlsbad, Calif., USA), BSA Fraction V (Thermo Fisher Scientific, Carlsbad, Calif., USA), and Glutamax (Thermo Fisher Scientific, Carlsbad, Calif., USA), or equivalents thereof. In some embodiments, the basal medium of the reversion media comprises each of DMEM/F12-Glutamax, Neurobasal medium, N2 supplement (Thermo Fisher Scientific, Carlsbad, Calif., USA), B27 supplement (Thermo Fisher Scientific, Carlsbad, Calif., USA), BSA Fraction V (Thermo Fisher Scientific, Carlsbad, Calif., USA), and Glutamax (Thermo Fisher Scientific, Carlsbad, Calif., USA), mTeSR (Stem Cell Technologies, Vancouver, BC, Canada), StemFit (Ajinomoto Co., Tokyo, JP) or equivalents thereof.

In some embodiments, the DMEM/F12-Glutamax or equivalent thereof, is present in the basal media at between about 25% and about 75% of the volume of the basal media. In one embodiment, the DMEM/F12-Glutamax or equivalent thereof, is present in the basal media at about 50% of the volume of the reversion media. In some embodiments, the Neurobasal medium or equivalent thereof, is present in the reversion media at between about 25% and about 75% of the volume of the reversion media. In one embodiment, the Neurobasal medium or equivalent thereof is present in the reversion media at about 50% of the volume of the basal media.

In some embodiments, the N2 supplement or equivalent thereof is present in the basal media at between about 0.0005% and about 5% of the volume of the basal media. In one embodiment, the N2 supplement or equivalent thereof is present in the basal media at about 0.5% of the volume of the basal media.

In some embodiments, the B27 supplement or equivalent thereof is present in the basal media at between about 0.001% and about 10% of the volume of the basal media. In one embodiment, the B27 supplement or equivalent hereof is present in the basal media at about 1% of the volume of the basal media.

In some embodiments, the BSA Fraction V or equivalent thereof is present in the basal media at between about 0.00007% and about 0.07% (from a 7.5% solution) of the volume of the basal media. In one embodiment, the BSA Fraction V or equivalent thereof is present in the basal media at about 0.005% (from a 7.5% solution), by weight, of the basal media.

In some embodiments, the Glutamax or equivalent thereof is present in the basal media at between about 0.0005% and about 5% of the volume of the basal media. In one embodiment, the Glutamax or equivalent thereof is present in the basal media at about 1% of the volume of the basal media.

In some embodiments, the basal media can be aliquoted (e.g., 50-, 100-, or 200-mL volumes) and frozen for later use.

In some embodiments, the first conversion media comprises the basal media and further comprises at least one additive (i.e., agent), for example, bone morphogenetic protein 4 (BMP4), ascorbic acid (Vitamin C), a SMAD inhibitor, or any combination thereof. In some embodiments the first conversion media comprises BMP4, ascorbic acid, and a SMAD inhibitor. The Smad inhibitor can be any Smad inhibitor known to one of skill in the art including, for example, SB431542 or GW788388. In some embodiments, the Smad inhibitor is SB431542. In some embodiments, the first conversion media comprises no or substantially no LIF and/or agonist of a lysophosphatidic acid receptor (LPAR agonist).

In some embodiments, the first conversion media comprises DMEM/F12 Glutamax Medium (Thermo Fisher Scientific), Neurobasal Medium (Thermo Fisher Scientific), N-2 Supplement (Thermo Fisher Scientific), B-27 Supplement (Thermo Fisher Scientific), 100× Glutamax Supplement (Thermo Fisher Scientific) and BSA Fraction V (Thermo Fisher Scientific) with Pen/Strep and 2-Mercaptoethanol, and supplemented with BMP4 and AA, and optionally supplemented with SB431542.

In some embodiments, the second conversion media comprises DMEM/F12 Glutamax Medium (Thermo Fisher Scientific), Neurobasal Medium (Thermo Fisher Scientific), N-2 Supplement (Thermo Fisher Scientific), B-27 Supplement (Thermo Fisher Scientific), 100× Glutamax Supplement (Thermo Fisher Scientific) and BSA Fraction V (Thermo Fisher Scientific) with Pen/Strep and 2-Mercaptoethanol, and supplemented with BMP4, AA, LIF, and OMPT.

In some embodiments, the first conversion media is the only conversion media used. In other embodiments, the first conversion media is used in combination with a second conversion media. The first conversion media and the second conversion media can be used in combination, sequentially, or substantially sequentially. In some embodiments, when used in combination at least a portion of the first conversion media and the second conversion media are admixed together, before, during, or after adding to the cells.

In some embodiments, the second conversion media comprises the basal media and further comprises at least one additive agent), for example, LIF, an LPAR agonist, ascorbic acid, or any combination thereof. In some embodiments the first conversion media, LIF, an LPAR agonist, and ascorbic acid. In some embodiments, the LPAR agonist is 1-oleoyl-2-methyl-sn-glycero-3-phosphothionate (OMPT), lysophosphatidic acid (LPA), or any combination thereof. In some embodiments, the second conversion media comprises no or substantially no BMP4 and/or SMAD inhibitor.

In some embodiments, the cells are cultured for a period of time in an amount of reversion media before or after culturing in the first conversion media and/or second conversion media. In one embodiment, the cells are cultured for a period of time in an amount of reversion media before culturing in the first conversion media and second conversion media. In some embodiments, the reversion media comprises the basal media, BMP4, LIF, a LPAR agonist, ascorbic acid, or any combination thereof. WO 2016/179243, the disclosure of which is hereby incorporated by reference herein in its entirety, describes formulations for reversion media.

### Culture Conditions

The cells of the present disclosure can be cultured under any conditions known to those in the field. For example, any growth substrate may be used, for example, feeder cells (e.g., mouse embryonic feeders (MEFs) and mouse fibroblast STO cell transformed with murine LIF and neomycin resistance (SNL)), extracellular matrices (e.g., Matrigel®, Cultrex® BME PathClear, Geltrex®), gelatin, collagen, poly-lysine, poly-ornithine, fibronectin, vitronectin, or laminin, among others. In some embodiments, the cells are cultured on a layer of fibronectin. In some embodiments, the laminin is laminin-511, for example, recombinant laminin-511) (iMatrix, Clontech, Mountain View, Calif., USA). In one embodiment, the cells are cultured under feeder free conditions.

In some embodiments, the cells of the disclosure are cultured in conditions of 1-20% oxygen (O₂) and 5% carbon dioxide (CO₂). In some embodiments, the cells are cultured under hypoxic conditions (e.g., in the presence of less than 10% O₂). In some embodiments, the cells are cultured at about 37° C. In some embodiments, the cells can be cultured at about 37° C., 5% CO₂ and 10-20% O₂.

In some embodiments, the cells are cultured in hypoxic conditions for a period of time. For example, the cells may be cultured under normoxic conditions (˜20% O₂) for a period of time and then switched to hypoxic conditions, for example ˜5% O₂. In other embodiments, the cells may be cultured under normoxic conditions for a period of time and then switched to hypoxic conditions and culture in a media (e.g., the first conversion media or the second conversion media) for a period of time. In other embodiments, the cells may be cultured under normoxic conditions for a period of time and then switched to hypoxic conditions and cultured in a media (e.g., the first conversion media or the second conversion media) for a period of time and then switched back to normoxic conditions in either the first conversion media or the second conversion media. In yet other embodiments, the cells may be cultured under hypoxic conditions in the first conversion media for a period of time then cultured in the second conversion media while maintaining the hypoxic conditions.

### Methods

Aspects of the present disclosure provide methods of deriving totipotent and/or totipotent like cells. In one aspect the disclosure provides in vitro methods of producing mammalian totipotent or totipotent like stem cells comprising: (a) obtaining a cell population of non totipotent cells and (b) providing the cell population with an amount of a first conversion media, thereby producing mammalian totipotent or totipotent-like stem cells.

In some embodiments, the cells are cultured in the first conversion media for about 6 hours to about 30 days, about 12 hours to about 20 days, about 1 day to about 10 days, about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 20 days, about 25 days, about 30 days, or more. In one embodiment, the cells are cultured in the first conversion media for about 4 days.

In one embodiment, the methods further comprise culturing the cells with an amount of a second conversion media. In some embodiments, the second conversion media is provided to the cells after the first conversion media. In one embodiment, the cells are cultured in the second conversion media for about 6 hours to about 30 days, about 12 hours to about 20 days, about 1 day to about 10 days, about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 20 days, about 25 days, about 30 days, or more. In one embodiment, the cells are cultured in the first conversion media for about 1 day to about 5 days. In another embodiment, the cells are cultured in the second conversion media for about 3 days.

In some embodiments, the cells are cultured in the first conversion media for about 1 day followed by culturing the cells in the second conversion media for about 6 hours to about 30 days, about 12 hours to about 20 days, about 1 day to about 10 days, about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 20 days, about 25 days, about 30 days, or more.

In some embodiments, the cells are cultured in the first conversion media for about 2 days followed by culturing the cells in the second conversion media for about 6 hours to about 30 days, about 12 hours to about 20 days, about 1 day to about 10 days, about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 20 days, about 25 days, about 30 days, or more.

In some embodiments, the cells are cultured in the first conversion media for about 3 days followed by culturing the cells in the second conversion media for about 6 hours to about 30 days, about 12 hours to about 20 days, about 1 day to about 10 days, about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 20 days, about 25 days, about 30 days, or more.

In some embodiments, the cells are cultured in the first conversion media for about 4 days followed by culturing the cells in the second conversion media for about 6 hours to about 30 days, about 12 hours to about 20 days, about 1 day to about 10 days, about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 20 days, about 25 days, about 30 days, or more.

In some embodiments, the cells are cultured in the first conversion media for about 5 days followed by culturing the cells in the second conversion media for about 6 hours to about 30 days, about 12 hours to about 20 days, about 1 day to about 10 days, about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 20 days, about 25 days, about 30 days, or more.

In some embodiments, the cells are cultured in the first conversion media for about 6 days followed by culturing the cells in the second conversion media for about 6 hours to about 30 days, about 12 hours to about 20 days, about 1 day to about 10 days, about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 20 days, about 25 days, about 30 days, or more.

In some embodiments, the cells are cultured in the first conversion media for about 7 days followed by culturing the cells in the second conversion media for about 6 hours to about 30 days, about 12 hours to about 20 days, about 1 day to about 10 days, about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 20 days, about 25 days, about 30 days, or more.

In some embodiments, the cells are cultured in the first conversion media for about 8 days followed by culturing the cells in the second conversion media for about 6 hours to about 30 days, about 12 hours to about 20 days, about 1 day to about 10 days, about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 20 days, about 25 days, about 30 days, or more.

In some embodiments, the cells are cultured in the first conversion media for about 9 days followed by culturing the cells in the second conversion media for about 6 hours to about 30 days, about 12 hours to about 20 days, about 1 day to about 10 days, about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 20 days, about 25 days, about 30 days, or more.

In some embodiments, the cells are cultured in the first conversion media for about 10 days followed by culturing the cells in the second conversion media for about 6 hours to about 30 days, about 12 hours to about 20 days, about 1 day to about 10 days, about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 20 days, about 25 days, about 30 days, or more.

In some embodiments, the cells are cultured in the first conversion media for about 11 days followed by culturing the cells in the second conversion media for about 6 hours to about 30 days, about 12 hours to about 20 days, about 1 day to about 10 days, about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 20 days, about 25 days, about 30 days, or more.

In some embodiments, the cells are cultured in the first conversion media for about 12 days followed by culturing the cells in the second conversion media for about 6 hours to about 30 days, about 12 hours to about 20 days, about 1 day to about 10 days, about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 20 days, about 25 days, about 30 days, or more.

In some embodiments, the cells are cultured in the first conversion media for about 13 days followed by culturing the cells in the second conversion media for about 6 hours to about 30 days, about 12 hours to about 20 days, about 1 day to about 10 days, about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 20 days, about 25 days, about 30 days, or more.

In some embodiments, the cells are cultured in the first conversion media for about 14 days followed by culturing the cells in the second conversion media for about 6 hours to about 30 days, about 12 hours to about 20 days, about 1 day to about 10 days, about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 20 days, about 25 days, about 30 days, or more.

In some embodiments, the cells are cultured in the first conversion media for about 15 days followed by culturing the cells in the second conversion media for about 6 hours to about 30 days, about 12 hours to about 20 days, about 1 day to about 10 days, about 1 day, about 2 days, about 3 days, about 4 days, about 5 days, about 6 days, about 7 days, about $ days, about 9 days, about 10 days, about 11 days, about 12 days, about 13 days, about 14 days, about 15 days, about 20 days, about 25 days, about 30 days, or more.

In some embodiments, the cells are cultured in the first conversion media for about 20 days followed by culturing the cells in the second conversion media for about 6 hours to about 30 days, about 12 hours to about 20 days, about 1 day to about 10 days, about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 20 days, about 25 days, about 30 days, or more.

In some embodiments, the cells are cultured in the first conversion media for about 25 days followed by culturing the cells in the second conversion media for about 6 hours to about 30 days, about 12 hours to about 20 days, about 1 day to about 10 days, about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 20 days, about 25 days, about 30 days, or more.

In some embodiments, the cells are cultured in the first conversion media for about 30 days followed by culturing the cells in the second conversion media for about 6 hours to about 30 days, about 12 hours to about 20 days, about 1 day to about 10 days, about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 20 days, about 25 days, about 30 days, or more.

In one embodiment, the methods comprise producing a blastocyst-like hemisphere. In some embodiments, the blastocyst-like hemisphere is produced within about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 20 days, about 30 days, about 40 days, about 50 days, or more when cultured in the first conversion media, the second conversion media, or both. In some embodiments, the blastocyst-like hemisphere is produced within about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 20 days, about 30 days, about 40 days, about 50 days, or more when cultured in the first conversion media. In some embodiments, the blastocyst-like hemisphere is produced within about 1 day, about 2 days, about 3 days, about 4 days, about 5 days, about 6 days, about 7 days, about 8 days, about 9 days, or about 10 days when cultured in the first conversion media. In one embodiment, the methods for producing a blastocyst like hemisphere include adding an incremental increase in the concentration of Smad inhibitor in the first conversion media, for example, using an initial amount of Smad inhibitor of between about 1 μM and about 3 μM and a second amount (or plurality of additional amounts) of Smad inhibitor of between about 2 μM and about 10 μM. In one embodiment an initial amount of Smad inhibitor at about 1 μM is used on the first day of conversion and 3 μM of Smad inhibitor is used on the next three days of conversion. In some embodiments, Smad inhibitors are expressly excluded from methods for producing a blastocyst-like hemisphere.

In one embodiment, the methods comprise producing an induced blastocyst-like structure (iBC). In some embodiments, the iBCs are also implantation-competent blastocyst-like structures. In some embodiments, the iBCs and/or implantation-competent blastocyst-like structures are characterized by one or more of a blastocoel-like cavity, outer cells positive for at least one trophectoderm lineage marker, and inner cells positive for at least one pluripotency marker. In some embodiments, the iBC is produced after culturing non totipotent cells in the first conversion media for a period of time, for example, about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 20 days, about 30 days, about 40 days, about 50 days, or more and cultured in the second conversion media for about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 20 days, about 30 days, about 40 days, about 50 days, or more. In one embodiment, the methods for producing iBCs includes adding an incremental change (e.g., an increase or decrease) in the concentration of Smad inhibitor in the first conversion media, for example, an using initial amount of Smad inhibitor of between about 1 μM and about 3 μM and a second amount (or plurality of additional amounts) of Smad inhibitor of between about 2 μM and about 10 μM. In one embodiment an initial amount of Smad inhibitor at about 1 μM is used on the first day of conversion and 3 μM of Smad inhibitor is used on the next three days of conversion.

In some embodiments, the cells (e.g., human iBCs or human blastocyst-like hemisphere), are grown for 14 days or less. In some embodiments, the cells are grown until just prior to formation of the primitive streak. One of skill in the art is readily able to identify development of a primitive streak by, for example, microscopy.

In some embodiments, at least 0.0005%, at least 0.05%, at least 0.005%, at least 0.5%, at least 1%, at least 5%, or more of the non-totipotent cells are converted to totipotent or totipotent-like stem cells. In some embodiments, greater than about 5%, greater than about 10%, greater than about 20%, greater than about 25%, greater than about 30%, greater than about 35%, greater than about 40%, greater than about 45%, greater than about 50%, greater than about 55%, greater than about 60%, greater than about 65%, greater than about 70%, greater than about 75%, greater than about 80%, greater than about 85%, greater than about 90%, greater than about 95%, greater than about 97%, greater than about 99%, or 100% of the non-totipotent cells are converted to totipotent or totipotent like stem cells.

In some embodiments, the non-totipotent cells are converted to totipotent and/or totipotent-like stem cells within about 1 day, about 2 days, about 3 days, about 4 days, about 5 days, about 6 days, about 7 days, about 8 days, about 9 days, about 10 days, about 15 days, about 20 days, about 30 days, about 40 days, about 50 days, about 60 days, about 70 days, about 80 days, about 90 days, about 100 days. In one embodiment, the non totipotent cells are converted to totipotent and/or totipotent-like stem cells within about 40 days. In one embodiment, the non-totipotent cells are converted to totipotent and/or totipotent-like stem cells within about 20 days. In one embodiment, the non-totipotent cells are converted to totipotent and/or totipotent-like stem cells within about 10 days.

The iBCs of the present disclosure can express one or a plurality of blastocyst markers. Non-limiting examples of blastocyst markers include, for example, Troma-I, Oct4, nuclear Cdx2, YAP, or any combination thereof. In some embodiments, the iBCs of the present disclosure differ from natural BCs (BCs) in at least one characteristic that defines natural BCs. For example, single-cell (sc) RNA sequencing or RNA-sequencing (RNA-seq) may be useful for integrating gene expression. Non-limiting examples of genes that can differ include, Troma-I, Oct4, Nanog, Sox2, Gata4, Sox17, Ecadherin, Eomes, Cripto, nuclear Cdx2, YAP, trophoblast markers (e.g., trophoblast specific protein A (TPBPA) and placental lactogen 1 (PL-1)), Zfp42(Rex1), Sox2, Zscan4, and other naïve pluripotency transcription factors in the cells of the inner cell mass and/or trophectoderm. In some embodiments, the genes may be expressed in iBCs but at a lower level compared to BCs. In some embodiments, the genes may be expressed in iBCs but at a higher level compared to BCs. In one embodiment, iBCs expressed more Troma-I and/or less Oct4 compared to BCs.

In some embodiments, the iBCs have a lower decidualization response as compared to BCs, for example, iBCs derive decidua less than about 80%, less than about 70%, less than about 60%, less than about 50%, less than about 40%, less than about 30%, less than about 20%, or less than about 10% of the time, while BCs derive decidua with greater than 80% efficacy and typically closer to 100% efficiency.

In other embodiments, the deciduae derived from the iBCs are focal and/or recruit blood vessels.

In some embodiments, at least a portion of the methods of the present disclosure are performed in vitro. In some embodiments, at least a portion of the methods of the present disclosure are performed in vivo. In some embodiments, a portion of the methods of the present disclosure are performed in vitro while another portion of the methods are performed in vivo. In some embodiments, at least a portion of the steps are carried out on a cell attachment substrate. In some embodiments, the methods comprise culturing the cells in a low attachment plate.

In some embodiments the iBC is an isogenic iBC. In some embodiments, in vitro formation of the totipotent and/or totipotent-like cell from the non-totipotent cell does not require formation of gametes (e.g., sperm or egg). In some embodiments, use of a sperm or and egg is expressly disclaimed.

The methods provided herein, further comprise transplanting the iBC into an animal, for example, a mouse (e.g., a pseudopregant mouse). In some embodiments, the iBCs induces partial or complete decidualization.

Aspects of the disclosure also provide methods of maintaining mammalian totipotent or totipotent-like cells in vitro, including for example, blastocyst-like hemispheres and/or iBCs. In some embodiments, the mammalian totipotent or totipotent-like cells are maintained in vitro for about 1 day, about 2 days, about 3 days, about 4 days, about 5 days, about 6 days, about 7 days, about 8 days, about 9 days, about 10 days, about 15 days, about 20 days, about 30 days, about 40 days, about 50 days, about 60 days, about 70 days, about 80 days, about 90 days, about 100 days, about 200 days, about 300 days, about a year, about 2 years, about 3 years, about 4 years, about 5 years, or longer.

In some embodiments, MERVL positive cells arise in the culture and can be seen for about 1 to about 50 days, about 1 to about 40 days, about 1 to about 30 days, about 1 to about 20 days, about 1 to about 10 days, about 9 days, about 8 days, about 7 days, about 6 days, about 5 days, about 4 days, about 3 days, about 2 days, about 1 day, or less. In some embodiments, MERVL positive cells arise in the culture and can be seen for about 1 to about 3 days. In some embodiments, greater than about 5%, greater than about 10%, greater than about 15%, greater than about 20%, greater than about 25%, greater than about 30%, greater than about 35%, greater than about 40%, greater than about 45%, greater than about 50%, greater than about 55%, greater than about 60%, greater than about 65%, greater than about 70%, greater than about 75%, greater than about 80%, greater than about 85%, greater than about 90%, greater than about 95% of the cells in culture are positive for MERVL expression. In some embodiments, all or substantially all of the cells in culture are positive for MERVL expression.

In some embodiments, MERVL positive cells arise in the culture and can be seen for about 1 to about 50 days in an amount of greater than about 5%, greater than about 10%, greater than about 15%, greater than about 20%, greater than about 25%, greater than about 30%, greater than about 35%, greater than about 40%, greater than about 45%, greater than about 50%, greater than about 55%, greater than about 60%, greater than about 65%, greater than about 70%, greater than about 75%, greater than about 80%, greater than about 85%, greater than about 90%, greater than about 95% of the cells in culture. In some embodiments, all or substantially all of the cells in culture are positive for MERVL expression.

In some embodiments, MERVL positive cells arise in the culture and can be seen for about 1 to about 40 days in an amount greater than about 5%, greater than about 10%, greater than about 15%, greater than about 20%, greater than about 25%, greater than about 30%, greater than about 35%, greater than about 40%, greater than about 45%, greater than about 50%, greater than about 55%, greater than about 60%, greater than about 65%, greater than about 70%, greater than about 75%, greater than about 80%, greater than about 85%, greater than about 90%, greater than about 95% of the cells in culture. In some embodiments, all or substantially all of the cells in culture are positive for MERVL expression.

In some embodiments, MERVL positive cells arise in the culture and can be seen for about 1 to about 30 days in amount greater than about 5%, greater than about 10%, greater than about 15%, greater than about 20%, greater than about 25%, greater than about 30%, greater than about 35%, greater than about 40%, greater than about 45%, greater than about 50%, greater than about 55%, greater than about 60%, greater than about 65%, greater than about 70%, greater than about 75%, greater than about 80%, greater than about 85%, greater than about 90%, greater than about 95% of the cells in culture. In some embodiments, all or substantially all of the cells in culture are positive for MERVL expression.

In some embodiments, MERVL positive cells arise in the culture and can be seen for about 1 to about 20 days in an amount greater than about 5%, greater than about 10%, greater than about 15%, greater than about 20%, greater than about 25?, greater than about 30%, greater than about 35%, greater than about 40%, greater than about 45%, greater than about 50%, greater than about 55%, greater than about 60%, greater than about 65%, greater than about 70%, greater than about 75%, greater than about 80%, greater than about 85%, greater than about 90%, greater than about 95% of the cells in culture. In some embodiments, all or substantially all of the cells in culture are positive for MERVL expression.

In some embodiments, MERVL positive cells arise in the culture and can be seen for about 1 to about 10 days in an amount greater than about 5%, greater than about 10%, greater than about 15%, greater than about 20%, greater than about 25%, greater than about 30%, greater than about 35%, greater than about 40%, greater than about 45%, greater than about 50%, greater than about 55%, greater than about 60%, greater than about 65%, greater than about 70%, greater than about 75%, greater than about 80%, greater than about 85%, greater than about 90%, greater than about 95% of the cells in culture. In some embodiments, all or substantially all of the cells in culture are positive for MERVL expression.

In some embodiments, MERVL positive cells arise in the culture and can be seen for about 9 days in an amount greater than about 5%, greater than about 10%, greater than about 15%, greater than about 20%, greater than about 25%, greater than about 30%, greater than about 35%, greater than about 40%, greater than about 45%, greater than about 50%, greater than about 55%, greater than about 60%, greater than about 65%, greater than about 70%, greater than about 75%, greater than about 80%, greater than about 85%, greater than about 90%, greater than about 95% of the cells in culture. In some embodiments, all or substantially all of the cells in culture are positive for MERVL expression.

In some embodiments, MERVL positive cells arise in the culture and can be seen for about 8 days in an amount greater than about 5%, greater than about 10%, greater than about 15%, greater than about 20%, greater than about 25%, greater than about 30%, greater than about 35%, greater than about 40%, greater than about 45%, greater than about 50%, greater than about 55%, greater than about 60%, greater than about 65%, greater than about 70%, greater than about 75%, greater than about 80%, greater than about 85%, greater than about 90%, greater than about 95% of the cells in culture. In some embodiments, all or substantially all of the cells in culture are positive for MERVL expression.

In some embodiments, MERVL positive cells arise in the culture and can be seen for about 7 days in an amount greater than about 5%, greater than about 10%, greater than about 15%, greater than about 20%, greater than about 25%, greater than about 30%, greater than about 35%, greater than about 40%, greater than about 45%, greater than about 50%, greater than about 55%, greater than about 60%, greater than about 65%, greater than about 70%, greater than about 75%, greater than about 80%, greater than about 85%, greater than about 90%, greater than about 95% of the cells in culture. In some embodiments, all or substantially all of the cells in culture are positive for MERVL expression.

In some embodiments, MERVL positive cells arise in the culture and can be seen for about 6 days in an amount greater than about 5%, greater than about 10%, greater than about 15%, greater than about 20%, greater than about 25%, greater than about 30%, greater than about 35%, greater than about 40%, greater than about 45%, greater than about 50%, greater than about 55%, greater than about 60%, greater than about 65%, greater than about 70%, greater than about 75%, greater than about 80%, greater than about 85%, greater than about 90%, greater than about 95% of the cells in culture. In some embodiments, all or substantially all of the cells in culture are positive for MERVL expression.

In some embodiments, MERVL positive cells arise in the culture and can be seen for about 5 days in an amount greater than about 5%, greater than about 10%, greater than about 15%, greater than about 20%, greater than about 25%, greater than about 30%, greater than about 35%, greater than about 40%, greater than about 45%, greater than about 50%, greater than about 55%, greater than about 60%, greater than about 65%, greater than about 70%, greater than about 75%, greater than about 80%, greater than about 85%, greater than about 90%, greater than about 95% of the cells in culture. In some embodiments, all or substantially all of the cells in culture are positive for MERVL expression.

In some embodiments, MERVL positive cells arise in the culture and can be seen for about 4 days in an amount greater than about 5%, greater than about 10%, greater than about 15%, greater than about 20%, greater than about 25%, greater than about 30%, greater than about 35%, greater than about 40%, greater than about 45%, greater than about 50%, greater than about 55%, greater than about 60%, greater than about 65%, greater than about 70%, greater than about 75%, greater than about 80%, greater than about 85%, greater than about 90%, greater than about 95% of the cells in culture. In some embodiments, all or substantially all of the cells in culture are positive for MERVL expression.

In some embodiments, MERVL positive cells arise in the culture and can be seen for about 3 days in an amount greater than about 5%, greater than about 10%, greater than about 15%, greater than about 20%, greater than about 25%, greater than about 30%, greater than about 35%, greater than about 40%, greater than about 45%, greater than about 50%, greater than about 55%, greater than about 60%, greater than about 65%, greater than about 70%, greater than about 75%, greater than about 80%, greater than about 85%, greater than about 90%, greater than about 95% of the cells in culture. In some embodiments, all or substantially all of the cells in culture are positive for MERVL expression.

In some embodiments, MERVL positive cells arise in the culture and can be seen for about 2 days in an amount greater than about 5%, greater than about 10%, greater than about 15%, greater than about 20%, greater than about 25%, greater than about 30%, greater than about 35%, greater than about 40%, greater than about 45%, greater than about 50%, greater than about 55%, greater than about 60%, greater than about 65%, greater than about 70%, greater than about 75%, greater than about 80%, greater than about 85%, greater than about 90%, greater than about 95% of the cells in culture. In some embodiments, all or substantially all of the cells in culture are positive for MERVL expression.

In some embodiments, MERVL positive cells arise in the culture and can be seen for about 1 day in an amount greater than about 5%, greater than about 10%, greater than about 15%, greater than about 20%, greater than about 25%, greater than about 30%, greater than about 35%, greater than about 40%, greater than about 45%, greater than about 50%, greater than about 55%, greater than about 60%, greater than about 65%, greater than about 70%, greater than about 75%, greater than about 80%, greater than about 85%, greater than about 90%, greater than about 95% of the cells in culture. In some embodiments, all or substantially all of the cells in culture are positive for MERVL expression.

In some embodiments, MERVL positive cells arise in the culture and can be seen for between about 2 and about 24 hours, about 3 and about 12 hours, about 4 and about 6 hours, more than about 30 minutes, more than about 1 hour, more than about 2 hours, more than about 3 hours, more than about 4 hours, more than about 5 hours, more than about 6 hours, more than about 7 hours, more than about 8 hours, more than about 9 hours, more than about 10 hours, more than about 11 hours, more than about 12 hours, more than about 13 hours, more than about 14 hours, more than about 15 hours, more than about 16 hours, more than about 17 hours, more than about 18 hours, more than about 19 hours, more than about 20 hours, more than about 21 hours, more than about 22 hours, more than about 23 hours, or more than about 24 hours in an amount greater than about 5%, greater than about 10%, greater than about 15%, greater than about 20%, greater than about 25%, greater than about 30%, greater than about 35%, greater than about 40%, greater than about 45%, greater than about 50%, greater than about 55%, greater than about 60%, greater than about 65%, greater than about 70%, greater than about 75%, greater than about 80%, greater than about 85%, greater than about 90%, greater than about 95% of the cells in culture. In some embodiments, all or substantially all of the cells in culture are positive for MERVL expression.

In some embodiments, MERVL positive cells arise in the culture and can be seen for about 1 to about 3 days. In some embodiments, greater than. about 5%, greater than about 10%, greater than about 15%, greater than about 20%, greater than about 25%, greater than about 30%, greater than about 35%, greater than about 40%, greater than about 45%, greater than about 50%, greater than about 55%, greater than about 60%, greater than about 65%, greater than about 70%, greater than about 75%, greater than about 80%, greater than about 85%, greater than about 90%, greater than about 95% of the cells in culture are positive for MERVL expression. In some embodiments, all or substantially all of the cells in culture are positive for MERVL expression.

In some aspects provided herein is an isolated totipotent or totipotent-like cell prepared according to any method of the present disclosure. In some embodiments, the isolated totipotent or totipotent-like cells are aggregates of isolated totipotent and/or totipotent-like cells. In some embodiments, the aggregate comprises a 2-cell, 4-cell, 8-cell, 16-cell, 32-cell, 64-cell aggregate of totipotent or totipotent like stem cells. In some embodiments, the aggregate comprises a 2-cell, 4-cell, 8-cell, or 16-cell aggregate of totipotent or totipotent-like cells.

Also provided herein are methods of producing tissue, organoids, and/or organs from the in vitro derived totipotent or totipotent-like cells produced according to an embodiment disclosed and described here. In some embodiments, the tissue or organ is a patient-specific tissue or organ. In some embodiments, at least a portion of the steps for producing tissue and/or organs are performed in vivo. In some embodiments, at least a portion of the steps for producing tissue and/or organs are performed in vitro.

In some aspects, provided herein are compositions of comprising induced totipotent and/or totipotent-like cells prepared by any one of the methods of using any of the media disclosed and described above. In some embodiments, the compositions comprise induced totipotent and/or totipotent-like cells and a pharmaceutical acceptable excipient.

The composition can comprise a pharmaceutically acceptable excipient, a pharmaceutically acceptable salt, diluents, carriers, vehicles and such other inactive agents well known to the skilled artisan. Vehicles and excipients commonly employed in pharmaceutical preparations include, for example, talc, gum Arabic, lactose, starch, magnesium stearate, cocoa butter, aqueous or non-aqueous solvents, oils, paraffin derivatives, glycols, etc. Solutions can be prepared using water or physiologically compatible organic solvents such as ethanol, 1,2-propylene glycol, polyglycols, dimethylsulfoxide, fatty alcohols, triglycerides, partial esters of glycerine and the like. Parenteral compositions may be prepared using conventional techniques that may include sterile isotonic saline water, 1,3-butanediol, ethanol, 1,2-propylene glycol, polyglycols mixed with water, Ringer's solution, etc.

Compositions may include a preservative and/or a stabilizer. Non-limiting examples of preservatives include methyl-, ethyl-, propyl-parabens, sodium benzoate, benzoic acid, sorbic acid, potassium sorbate, propionic acid, benzalkonium chloride, benzyl alcohol, thimerosal, phenylmercurate salts, chlorhexidine, phenol, 3-cresol, quaternary ammonium compounds (QACs), chlorbutanol, 2-ethoxyethanol, and imidurea.

In some embodiments, the composition may include a cryoprotectant agent. Non-limiting examples of cryoprotectant agents include a glycol (e.g., ethylene glycol, propylene glycol, and glycerol), dimethyl sulfoxide (DMSO), formamide, sucrose, trehalose, dextrose, and any combinations thereof.

### Kits

Also disclosed are kits comprising: (a) at least a first conversion media for converting a non-totipotent cell into a totipotent and/or totipotent-like cell, the media comprising BMP4, ascorbic acid, a Smad inhibitor, or any combination thereof; and (b) instructions. In some embodiments, the kits further comprise (b) a second conversion media comprising LIF, an LPAR agonist, ascorbic acid, or any combination thereof. In other embodiments, the kits further comprise isolated non-totipotent cells.

In some embodiments the kits comprise: (a) at least a first conversion media for converting a non-totipotent cell into a totipotent and/or totipotent-like cell, the media comprising BMP4, ascorbic acid, and a Smad inhibitor; and (b) instructions. In some embodiments, the kits further comprise (b) a second conversion media comprising LIF, an LPAR agonist, and ascorbic acid. In other embodiments, the kits further comprise isolated non-totipotent cells.

In some embodiments the kits comprise: (a) at least a first conversion media for converting a non-totipotent cell into a totipotent and/or totipotent-like cell, the media comprising BMP4, ascorbic acid, and SB43152; and (b) instructions. In some embodiments, the kits further comprise (b) a second conversion media comprising LIF, OMPT, and ascorbic acid. In other embodiments, the kits further comprise isolated non totipotent cells.

The components of the kit may be contained in one or different containers such as one or more vials. The cell culture media may be in liquid or solid form (e.g. after lyophilization) to enhance shelf-life. If in liquid form, the components may comprise additives that enhance shelf-life.

In various embodiments, instructions for use of the kits will include directions to use the kit components for converting a non-totipotent cell into a totipotent and/or totipotent-like cell. The instructions may further contain information regarding how to prepare (e.g., dilute, in the case of concentrated media) the media and the cells (e.g., thawing and/or culturing).

### EXAMPLE 1

### Production of 3D Blastocyst-Like Hemispheres from Epiblast Stem Cells

Mouse Epiblast Stem Cell (mEpiSC) Culture

Mouse EpiSCs used for conversion experiments were maintained as high quality culture conditions and expanded to a stock size large enough to support the conversion experiments. Cells were plated at 2-10% confluent on fibronectin-coated culture plates containing mEpiSC Culture Media (MCM) (NDiff227 Media (Clontech/Takara), Activin A at 20 ng/mL (Media Supplements), bFGF at 12 ng/mL (Media Supplements), and 100× penicillin/streptomycin solution). Media was changed daily, and cells passaged every 2-3 days at ˜1:10 to 1:20, never exceeding 30% confluent. Colonies were maintained to be less than 150 μM wide, on average, with cultures containing largely homogenous mEpiSC colonies with few singular cells. Cells were passaged using Accutase solution and a cell scraper and kept at as low passage as possible for conversion experiments.

### Mouse EpiSC Preparation for Conversion to 3D Blastocyst-Like Hemispheres

Plating mEpiSC as single cells for conversion to both blastocyst-like hemispheres and induced blastocyst-like cells required a large stock of mEpiSCs prepared as described above and careful treatment to remove less-desirable cells from culture. Single cells and colony-periphery cells were removed by incubating cells in Accutase solution for less than one minute. To detach the remaining mEpiSCs from the plate and separate from each other, the mEpiSCs were incubated in a fresh amount of Accutase for approximately 7-8 minutes at 37° C. Detached cells were collected in a solution of MCM:DPBS (1:1). Cells were spun and resuspended in MCM.

3D hemispheres were generated by plating approximately 20,000 mEpiSCs/fibronectin-coated well. Conversion began approximately 15-18 hours after incubation. On Conversion Day 0, cells were observed evenly dispersed as mostly single cells. After overnight incubation, cells mostly resembled evenly distributed single cells, with some forming 2 or 3 cell clusters. CTSFES media (DMEM/F12 Glutamax Medium (Thermo Fisher Scientific), Neurobasal Medium (Thermo Fisher Scientific), N-2 Supplement (Thermo Fisher Scientific), B-27 Supplement (Thermo Fisher Scientific), 100× Glutamax Supplement (Thermo Fisher Scientific) and 7.5% BSA Fraction V (Thermo Fisher Scientific) was supplemented with BMP4 (10 ng/mL), LIF (1000 units/mL), ascorbic acid (AA) (64 μg/mL), and OMPT (1 μM) for the first conversion media (also referred to herein as “Induction Media Phase 1,” “Phase 1” or the like). Blastocyst-like hemispheres were cultured in the first conversion media (changed daily) for about 7-8 days. Media was removed from 4° C. to room temperature 20-30 minutes before use and then store immediately after at 4° C.

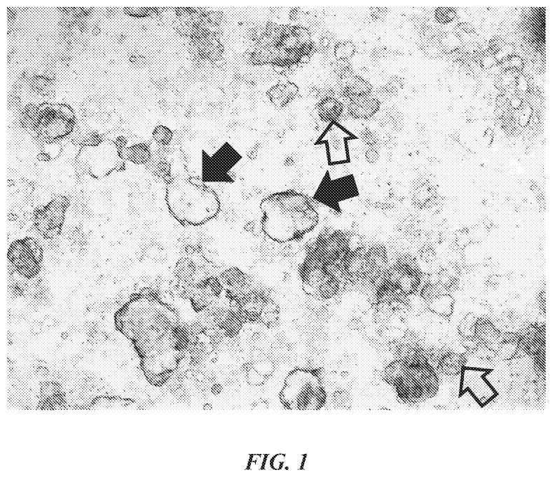

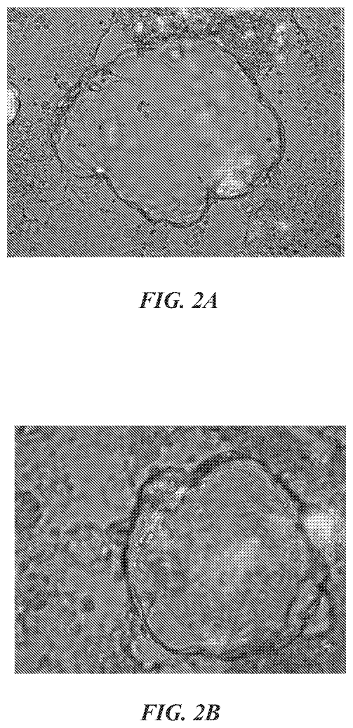

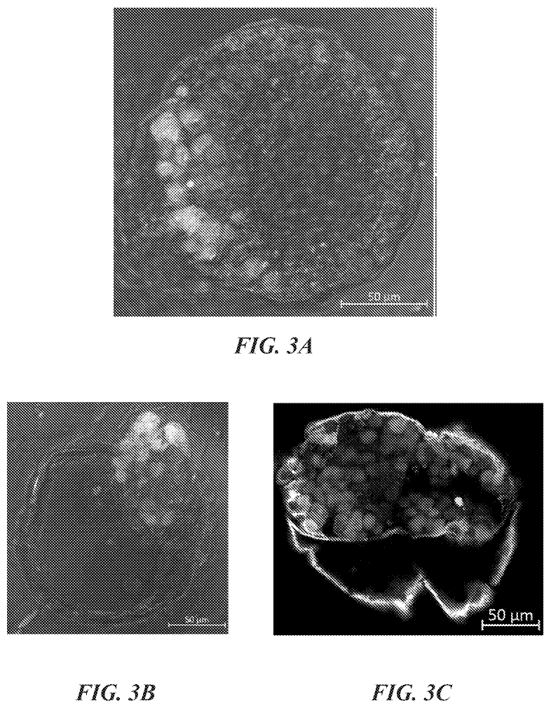

On about Day 7 or 8 of conversion, distinct domes of trophectoderm like cells emerged rapidly. At this time both morula-like and blastocyst like structures were observed to coexist on the plate, but generally contained more cells than expected in natural embryonic development. Morula-like structures appeared as refractive domes but were generally smaller than blastocyst-like hemispheres and were difficult to distinguish an early forming blastocoel-like space without confocal microscopy (FIG. 1, Open Arrows). Blastocyst-like hemispheres appeared larger and obvious with flat trophectoderm-like cells and one or two polar masses of naïve-like stem cells (FIG. 1, Closed Arrows and FIG. 2). FIG. 2 shows representative images of blastocyst-like hemispheres generated according to an embodiment of the present disclosure. Early blastocyst-like hemispheres expressed Nanog in non-flattened GFP negative cells (FIG. 3A), while Nanog expressed in late blastocyst-like hemispheres was restricted to the GFP positive cells (FIG. 3B). The blastocyst-like hemispheres also contained Troma-I (Krt8)-positive cells, a marker for trophectoderm lineage cells, surrounding the blastocoel-like space and oversized Xa/Xa-GFP positive, Nanog positive, polar mass. (FIG. 3C).

### EXAMPLE 2

### Production of Floating Pre-Implantation 3D Blastocyst-Like Decidua-Inducing Structures from Epiblast Stem Cells

Mouse EpiSC Conversion to Floating iBCs

Generation of sufficient numbers of mEpiSCs for iBC experiments were performed as described above for 3D Blastocyst-like Hemispheres generation, but by plating approximately 40,000-50,000 mEpiSCs/fibronectin-coated 9.8 cm² well.

Under the conditions tested, the conversion bias toward floating iBCs was less efficient; therefore, more cells were required than for the 3D hemispheres (discussed in example above) in order to observe or harvest iBCs. It is contemplated that optimization of timing and Smad inhibition may vary depending on the cell type used. As the Smad inhibition appeared to be toxic to starting mEpiSCs, the inhibitor concentration was slowly increased from 1 μM on Day 0 to 3 μM from Days 1-3. Two phases of media were prepared and cells were fed from days 0-3 (“first conversion media”) and then days 4-7 (“second conversion media”), respectively.

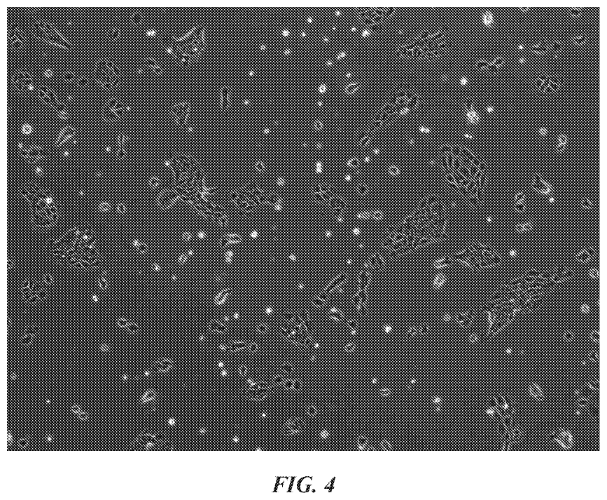

At Conversion Day 0, approximately 14 hours after preparation of mEpiSCs for conversion, cells were observed as evenly dispersed, mostly single cells. The first conversion media was prepared using CTSFES media with Pen/Strep and 2-Mercaptoethanol described above as the base media and supplemented with BMP4 (10 ng/mL), SB431542 (1 μM on Day 0, 3 μM from Days 1-3), and AA (64 μg/mL). On Conversion Day 1, cells were observed growing with some cells remaining colony-like while others began to flatten at the edges. (FIG. 4). SB431542 toxicity was also believed to be killing some cells. At approximately the same time each day, media was replaced and cells were incubated overnight at 37° C.

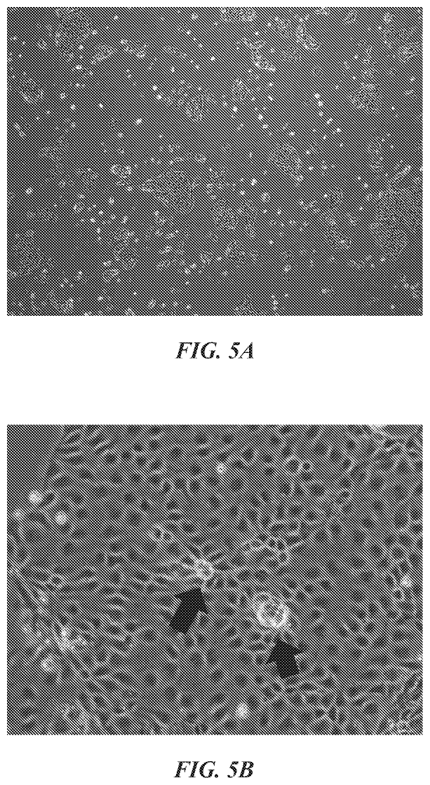

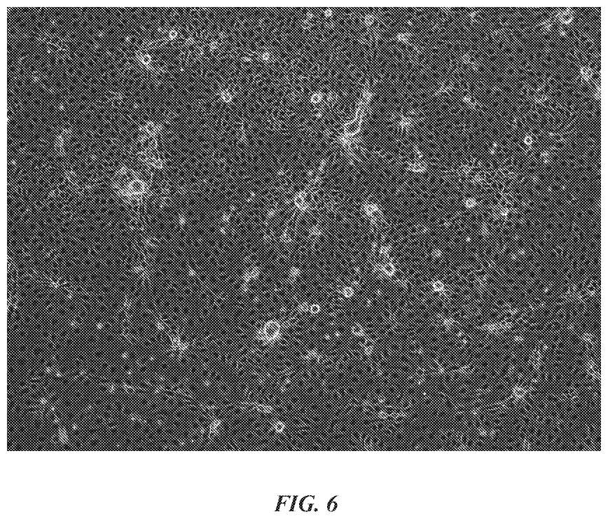

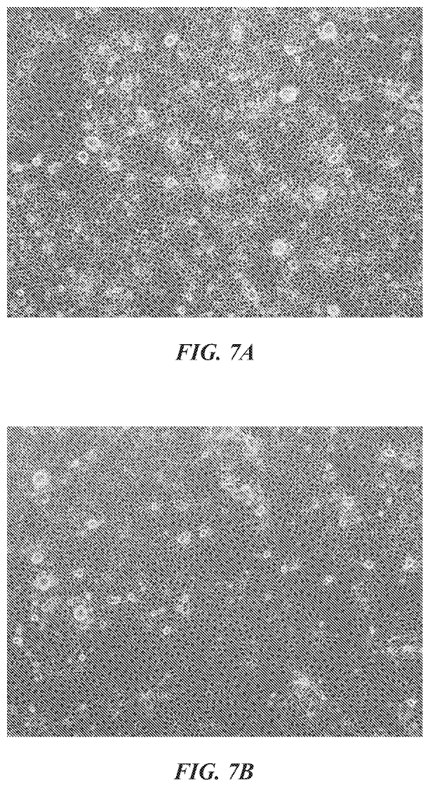

At Conversion Day 2, cells were observed to be flattening more rapidly at the edges (FIG. 5A) and clustering toward refractive grape-like clusters or pairs at margins (FIG. 5B, Arrows). At the same time of day as the prior conversion days, the first conversion media was aspirated from the plate and replenished with fresh first conversion media and incubated overnight at 37° C. At Conversion Day 3 cells were flattened or clustering toward refractive grape-like clusters as the plate approaches confluent (FIG. 6).

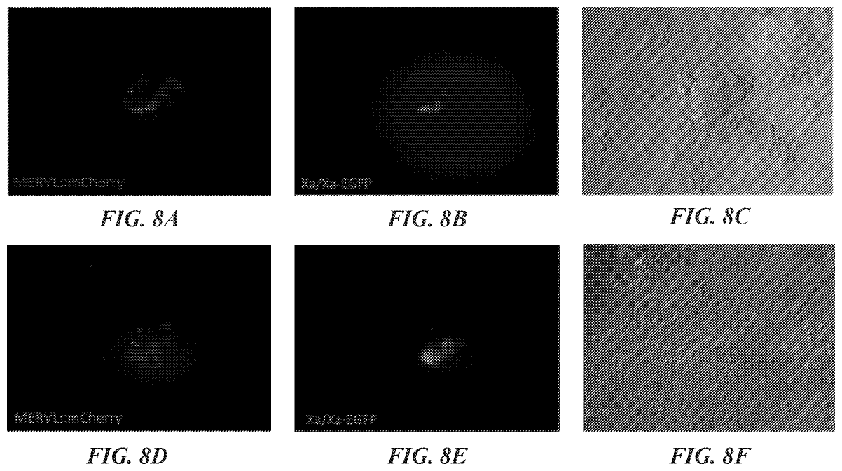

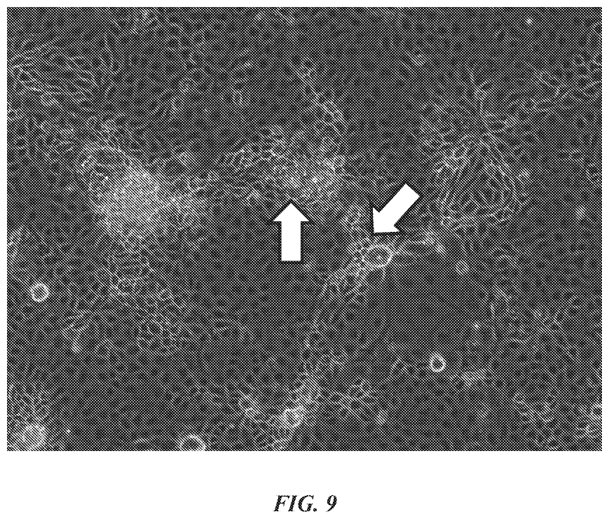

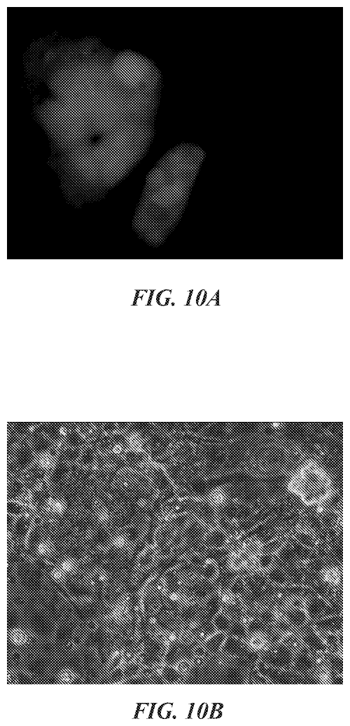

At Conversion Day 4, the first conversion media was replaced with a second conversion media prepared using CTSFES media with Pen/Strep and 2-Mercaptoethanol described above as the base media and supplemented with BMP4 (5 ng/mL), AA (64 μg/mL), LIF (500 units/mL) and OMPT (0.5 μM) and incubated overnight at 37° C. After 24 hours incubation in the second conversion media, cells continued to grow and grape-like clusters began to emanate compact light refractive spheres of cells (FIG. 7). In addition, co-expression of a totipotency reporter (MERVL) and Xa/Xa-GFP expression, colocalized were observed in the same cells by Conversion Day 4 (FIG. 8). Both MERVL) and Xa/Xa-GFP expression are hallmarks of totipotency. By Day 5, possible sources of totipotent-like or iBC forming clusters were observed (FIG. 9).

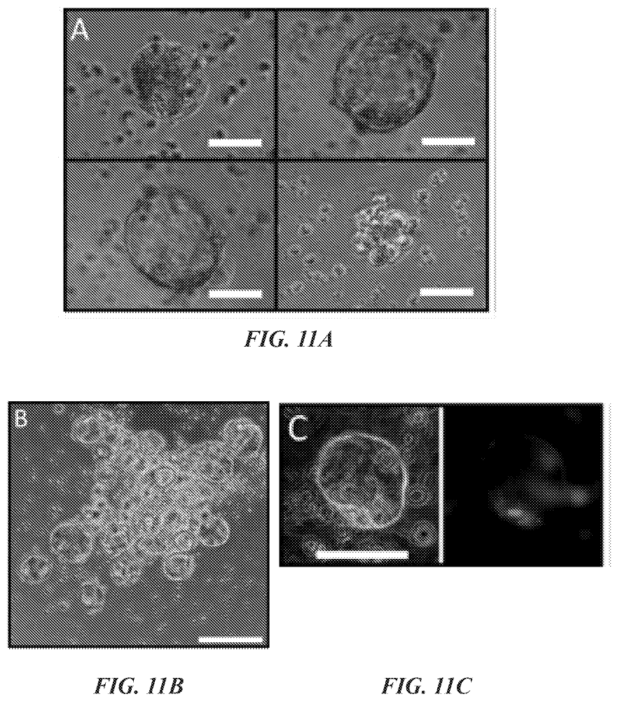

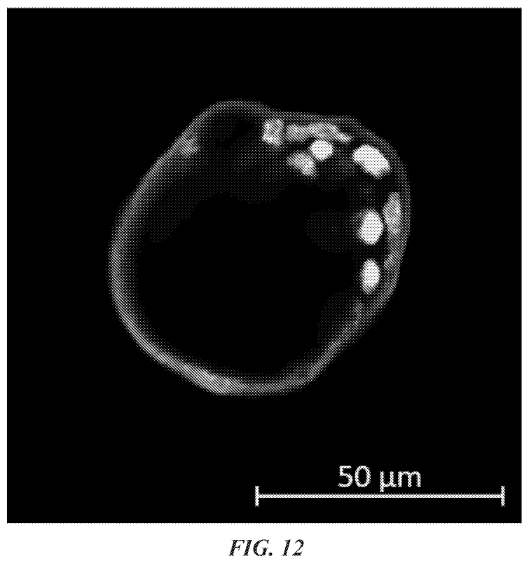

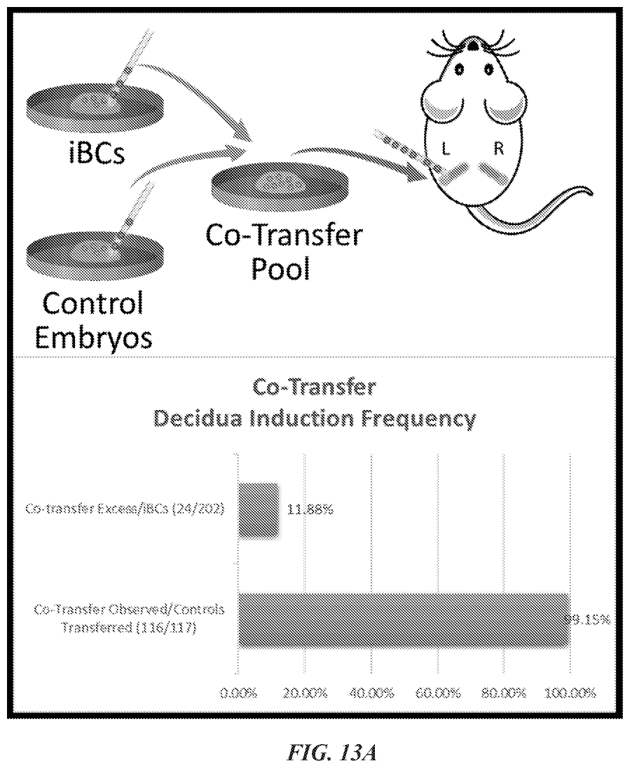

On Conversion Day 6, supernatants were collected in ultra-low attachment plates and then replaced with the second conversion media. The supernatant contained some cell clusters that form morula-like structures at this stage. The formation of the blastocoel like space at trophectoderm-like cells is a good predictor of successful iBC formation. The supernatants were replated onto an ultra-low attachment (ULA) plate. On Conversion Day 7, supernatants were again collected and replated to a ULA plate. After harvest, early blastocyst-like iBCs, were observed in the wells containing cells collected on Conversion Day 6 (FIG. 10). Induced iBCs were released into suspension. One to two days post-release, cells resembled late morula-like iBCs and appeared to pause at approximately 4 days post-release. At 5 days post-release sonic cells appeared as large 8 cell aggregates. (FIG. 11), and similar large cleavage-stage like MERVL-positive cells can be seen on the plate (FIG. 10). The iBC-like structures showed predictable blastocyst-like expression of the extraembryonic lineage marker (Troma-I) and a pluripotency marker (Oct3/4). (FIG. 12)

### EXAMPLE 3

### Implantation of iBCs Generate Decidua-Inducing Structures from Epiblast Stem Cells

To determine whether the iBCs described above retain characteristics of true blastocysts, iBCs were implanted into pseudopregnant female mice.

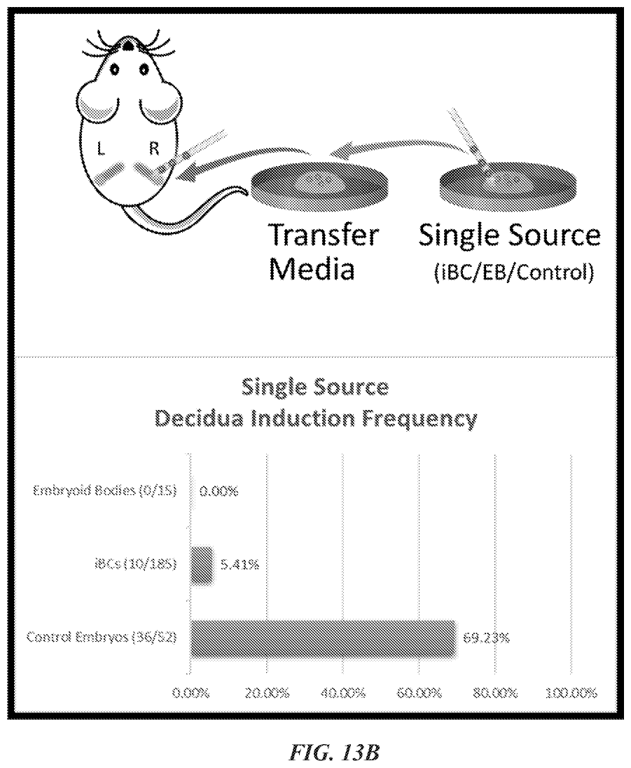

In these studies supernatants containing iBCs were collected and then observed for early blastocyst like morphology structures. Using 0.2-0.3 mm embryo pipettes, early blastocyst-like morphology structures were isolated to a pre-warmed pool of basal media. These optimal iBCs were then transferred with or without control embryos into the left or right uterus horns of pseudopregnant mice using standard in-vitro fertilization uterus transfer techniques. A schematic representation of the implantation experiments performed FIG. 13A shows an iBC and Control Embryo co-transfer experiment diagram.

When iBCs were co-transferred with control embryos, sometimes more decidua were observed than control embryos. Counting only the excess and dividing by the number of iBCs transferred in those experiments, this implies at least 11.8% success for iBCs. When counting all co-transfers, the total deciduae observed divided by the number of controls transferred showed 99.15%, while control-only transfers showed a frequency of 69.2%. (FIGS. 13A and B), suggesting iBCs contributed to observed deciduae in co-transfers. On the other hand, when iBCs were transferred alone, decidua induction was observed at a frequency of 5.41%, versus 0% when embryoid bodies from mEpiSC aggregation were transplanted. Again, a frequency of 69.2% was observed using control embryos alone. (FIG. 13B).

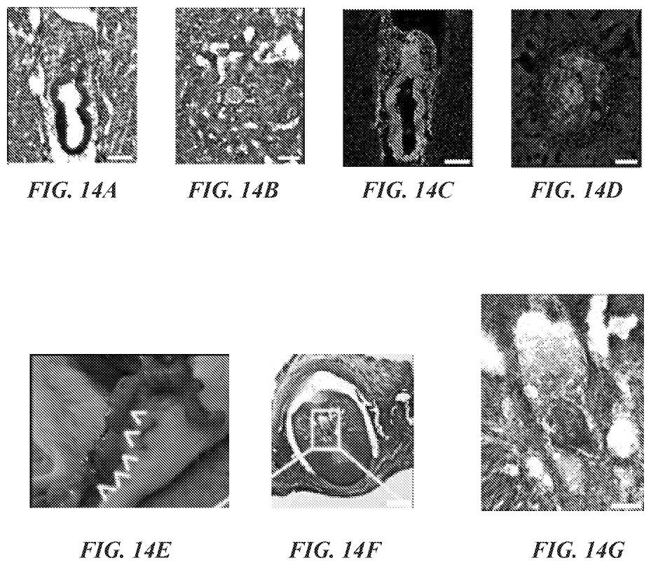

The iBCs induced decidualization and partially develop before resorption in utero. As shown by FIG. 14, embryos in deciduae for positive control H2B-EGFF E6.5 embryo. Co-transfer deciduae were positive control H2B-EGFP E6.5 embryo with anti-GFP antibody (green), Troma-I (magenta), and DNA (blue). In addition, excess decidua from co-transfer were observed with apparent non-decidua tissue which did not stain with anti-GFP antibody (green), but retained Troma-I positive cells(magenta), and DNA (blue) (FIGS. 14B and 14D). With iBC-only transfers to the uterus several induced deciduae at E7.5 were observed, dissected and prepared for cryosection and H&E stained to reveal decidua (FIG. 14E) with evidence of resorption including high presence of granulocytes and reduced embryonic cavity with disfigured trophectoderm and embryonic tissue morphology (FIGS. 14F and 14G). Together, these data suggest the iBCs of the present disclosure may be useful for generating isogenic mammals, without requiring donor blastocysts, gametes, and/or chimerism for extraembryonic support in utero.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains

In addition, where the features or aspects of the disclosure are described in terms of Markush groups, those skilled in the art will recognize that the invention is also thereby described in terms of any individual member or subgroup members of the Markush group

All publications, patent applications, patents and other references mentioned herein are expressly incorporated by reference in their entirety, to the same extent as if each were incorporated by reference individually. In case of conflict, the present disclosure, including definitions, will control.
1. Blaschke, K., Ebata, K. T., Karimi, M. M., Zepeda-Martinez, J. A., Goyal, P., Mahapatra, S., Tam, A., Laird, D. J., Hirst, M., Rao, A., et al. (2013). Vitamin C induces Tet-dependent DNA demethylation and a blastocyst-like state in ES cells. Nature 500, 222-226.
2. Cha, J., Sun, X., and Dey, S. K. (2012). Mechanisms of implantation: strategies for successful pregnancy. Nat Med 18, 1754-1767.
3. Chen, W., Jia, W., Wang, K., Zhou, Q., Leng, Y., Duan, T., and Kang, J. (2012). Retinoic acid regulates germ cell differentiation in mouse embryonic stem cells through a Smad-dependent pathway. Biochemical and Biophysical Research Communications 418, 571-577.
4. Cluing, T.-L., Brena, R. M., Kolle, G., Grimmond, S. M., Berman, B. P., Laird, P. W., Pera, M. F., and Wolvetang, E. J. (2010). Vitamin C Promotes Widespread Yet Specific DNA Demethylation of the Epigenome in Human Embryonic Stem Cells. STEM CELLS 28, 1848-1855.
5. Hübner, K., Fuhrmann, G., Christenson, L. K., Kehler, J., Reinbold, R., Fuente, R. D. L., Wood, J., Strauss, J. F., Boiani, M., and Schöler, H. R. (2003). Derivation of Oocytes from Mouse Embryonic Stem Cells. Science 300, 1251-1256.
6. Ishiuchi, T., Enriquez-Gasca, R., Mizutani, E., Bošković, A., Ziegler-Birling, C., Rodriguez-Terrones, D., Wakayama, T., Vaquerizas, J. M., and Torres-Padilla, M.-E. (2015). Early embryonic-like cells are induced by downregulating replication-dependent chromatin assembly. Nat Struct Mol Biol advance online publication.
7. Kime, C., Sakaki-Yumoto, M., Goodrich, L., Hayashi, Y., Sami, S., Derynck, R., Asahi, M., Panning, B., Yamanaka, S., and Tomoda, K. (2016). Autotaxin-mediated lipid signaling intersects with LIF and BMP signaling to promote the naive pluripotency transcription factor program. PNAS 113, 12478-12483.
8. Kimura, T., Kaga, Y., Sekita, Y., Fujikawa, K., Nakatani, T., Odamoto, M., Funaki, S., Ikawa, M., Abe, K., and Nakano, T. (2015). Pluripotent Stem Cells Derived From Mouse Primordial Germ Cells by Small Molecule Compounds. Stem Cells 33, 45-55.
9. Macfarlan, T. S., Gifford, W. D., Driscoll, S., Lettieri, K., Rowe, H. M., Bonanomi, D., Firth, A., Singer, O., Trono, D., and Pfaff, S. L. (2012). Embryonic stem cell potency fluctuates with endogenous retrovirus activity. Nature 487, 57-63.
10. Ozone, C., Suga, H., Eiraku, M., Kadoshima, T., Yonemura, S., Takata, N., Oiso, Y., Tsuji, T., and Sasai, Y. (2016). Functional anterior pituitary generated in self-organizing culture of human embryonic stem cells. Nat Commun 7, 10351.
11. Panciera, T., Azzolin, L., Fujimura, A., Di Biagio, D., Frasson, C., Bresolin, S., Soligo, S., Basso, G., Bicciato, S., Rosato A., et al. (2016). Induction of Expandable Tissue-Specific Stem/Progenitor Cells through Transient Expression of YAP/TAZ. Cell Stem Cell 19, 725-737.
12. Qin, H., Hejna, M., Liu, Y., Percharde, M., Wossidlo, M., Blouin, L., Durruthy-Durruthy, J., Wong, P., Qi, Z., Yu, J., et al. (2016). YAP Induces Human Naive Pluripotency. Cell Reports 14, 2301-2312.
13. Seydoux, G., and Braun, R. E. (2006). Pathway to Totipotency: Lessons from Germ Cells. Cell 127, 891-904.
14. Steward, F. C., Mapes, M. O., and Mears, K. (1958). Growth and Organized Development of Cultured Cells. II. Organization in Cultures Grown from Freely Suspended Cells. American Journal of Botany 45, 705-708.
15. Surani, M. A., and Hajkova, P. (2010). Epigenetic Reprogramming of Mouse Germ Cells toward Totipotency. Cold Spring Harb Symp Quant Biol sqb.2010.75.010.
16. Tang, F., Barbacioru, C., Bao, S., Lee, C., Nordman, F., Wang, X., Lao, K., and Surani, M. A. (2010). Tracing the Derivation of Embryonic Stem Cells from the Inner Cell Mass by Single-Cell RNA-Seq Analysis. Cell Stem Cell 6, 468-478.
17. Unno, N., Kuwabara, Y., Okai, T., Kido, K., Nakayama, H., Kikuchi, A., Narumiya, Y., Kozuma, S., Taketani, Y., and Tamura M. (1993). Development of an Artificial Placenta: Survival of Isolated Goat Fetuses for Three Weeks with Umbilical Arteriovenous Extracorporeal Membrane Oxygenation. Artificial Organs 17, 996-1003.
18. Wennekamp, S., Mesecke, S., Nédélec, F., and Hiiragi, T. (2013). A self-organization framework for symmetry breaking in the mammalian embryo. Nat Rev Mol Cell Biol 14, 452-459.
19. Wossidlo, M., Nakamura, T., Lepikhov, K., Marques, C. J., Zakhartchenko, V., Boiani, M., Arand, J., Nakano, T., Reik, W., and Walter, J. (2011). 5-Hydroxymethylcytosine in the mammalian zygote is linked with epigenetic reprogramming. Nature Communications 2, 241.
20. Wu, J., Huang, B., Chen, H., Yin, Q., Liu, Y., Xiang, Y., Zhang, B., Liu, B., Wang, Q., Xia, W., et al. (2016). The landscape of accessible chromatin in mammalian preimplantation embryos. Nature advance online publication.
21. Yamaji, M., Seki, Y., Kurimoto, K., Yabuta, Y., Yuasa, M., Shigeta, M., Yamanaka, K., Ohinata, Y., and Saitou, M. (2008). Critical function of Prdm14 for the establishment of the germ cell lineage in mice. Nat Genet 40, 1016-1022.
22. Ying, Q.-L., Nichols, J., Chambers, I., and Smith, A. (2003). BMP Induction of Id Proteins Suppresses Differentiation and Sustains Embryonic Stem Cell Self-Renewal in Collaboration with STAT3. Cell 115, 281-292.

1. An in vitro method of producing mammalian totipotent and/or totipotent-like stem cells comprising:
(a) obtaining a cell population of non-totipotent cells; and
(b) providing the cell population with an amount of a first conversion media,
thereby producing mammalian totipotent and/or totipotent-like stem cells from the non-totipotent cells.

2. The method of claim 1, wherein the non-totipotent cells are selected from the group consisting of a pluripotent stem cell, an epiblast stem cell, an adult stem cell, and a fibroblast, or a combination thereof.

3. The method of claim 2, wherein the pluripotent stem cell is a naive pluripotent stem cell or a primed pluripotent stem cell.

4. The method of claim 1, wherein the totipotent or totipotent-like stem cells can contribute to both extraembryonic and embryonic lineages.

5. The method of claim 1, wherein the totipotent or totipotent-like stem cells express Cdx2, YAP, Hex, Oct4, H3R2me2, Prdm14, H3K4me2, Mouse Retroelement MuERV-L/MERVL, XatXa-GFP, or any combination thereof.

6. The method of claim 1, wherein the mammalian totipotent or totipotent-like stem cells are human totipotent or totipotent-like stem cells.

7. The method of claim 1, wherein the mammalian totipotent or totipotent-like stem cells are human totipotent or totipotent-like stem cells.

8. The method of claim 1, wherein at least 0.005%, at least 0.05%, at least 0.5%, at least 1%, at least 5%, or more of the non-totipotent cells are converted to totipotent or totipotent-like stem cells.

9. The method of claim 1, wherein prior to step (b) the cell population are provided an amount of reversion media.

10. The method of claim 9, wherein the reversion media comprises BMP4, LIF, a LPAR agonist, ascorbic acid, or any combination thereof.

11. The method of claim 1, wherein the first conversion media comprises BMP4, ascorbic acid, a Smad inhibitor, or any combination thereof.

12. The method of claim 11, wherein the Smad inhibitor is SB431542.

13. The method of claim 11, wherein the first conversion media comprises no or substantially no LIF and/or LPAR agonist.

14. The method of claim 1, wherein the cell population is cultured in the first conversion media for about 1 to about 10 days.

15. The method of claim 11, wherein the cell population is cultured in the first conversion media for about 4 days.

16. The method of claim 1, further comprising culturing the cell population with an amount of a second conversion media.

17. The method of claim 16, wherein the second conversion media comprises LIF, an LPAR agonist, ascorbic acid, or any combination thereof.

18. The method of claim 16, wherein the second conversion media comprises no or substantially no BMP4 and/or SMAD inhibitor.

19. The method of claim 17, wherein the LPAR agonist is 1-oleoyl-2-methyl-sn-glycero-3-phosphothionate (OMPT), lysophosphatidic acid (LPA), or any combination thereof.

20. The method of claim 16, wherein the second conversion media is provided after the first conversion media.

21. The method of claim 16, wherein the second conversion media is provided for about 1 day to about 5 days.

22. The method of claim 21, wherein the second conversion media is provided for about 3 days.

23. The method of claim 1, further comprising producing a morula-like hemisphere from the mammalian totipotent and/or totipotent-like stem cells.

24. The method of claim 1, further comprising producing a blastocyst-like hemisphere from the mammalian totipotent and/or totipotent-like stem cells.

25. The method of claim 1, further comprising producing an induced blastocyst-like structure (iBC) from the mammalian totipotent and/or totipotent-like stem cells.

26. The method of claim 1, wherein cells of the iBC express Troma-I, Oct4, nuclear Cdx2, YAP, or any combination thereof.

27. The method of any one of claim 23, wherein the producing is in vitro or in vivo.

28. The method of claim 25, wherein the iBC is an isogenic iBC.

29. The method of claim 25, further comprising transplanting the iBC into a pseudopregnant mouse.

30. The method of claim 29, wherein the iBC induces decidualization.

31. The method of claim 1, wherein in at least a portion of the steps are carried out on a cell attachment substrate.

32. The method of claim 1, further comprising culturing the cells in a 1 mv attachment plate.

33. A method of maintaining mammalian totipotent or totipotent-like cells in vitro.

34. An isolated totipotent or totipotent-like cell prepared by any one of claim 1.

35. An aggregate of the isolated totipotent or totipotent-like cells of claim 34.

36. The aggregate of claim 35, wherein the aggregate is a 2-cell, 4-cell, 8-cell, 16-cell, 32-cell, 64-cell aggregate of totipotent or totipotent-like cells.

37. A method of producing tissue and/or organs from the in vitro derived totipotent or totipotent-like cells of claim 1.

38. The method of claim 37, wherein the tissue or organ is a patient-specific tissue or organ.

39. The method of claim 37, wherein at least a portion of the steps are performed in vitro.

40. The method of claim 37, wherein at least a portion of the steps are performed in vivo.
